(19) United States
(12) Patent Application Publication
(10) Pub. No.: US 2021/0145691 A1
(43) Pub. Date: May 20, 2021

(54) HEAD MASSAGE DEVICE

(71) Applicant: Chung-Sing LIN, Tainan (TW)

(72) Inventor: Chung-Sing LIN, Tainan (TW)

(73) Assignee: Chung-Sing LIN, Tainan (TW)

(21) Appl. No.: 16/948,943

(22) Filed: Oct. 6, 2020

(30) Foreign Application Priority Data
Nov. 19, 2019 (TW) 108141856

(51) Int. Cl. A61H 23/02

(52) U.S. Cl. CPC A61H 23/0218; A61H 2201/0153; A61H 2201/105; A61H 2205/02

(57) ABSTRACT

A head massage device includes a head with a tubular member extending from the top thereof. A needle extends from the inner bottom of the tubular member. Multiple first bores are defined through the inner bottom of the tubular member. A handle is connected to the head and has a vibrator received therein which is controlled by a switch. A magnetic part is connected to the underside of the head and has protrusions extending from the underside thereof. Multiple holes are defined through the magnetic part, and each hole communicates with the passage of the protrusion located corresponding thereto. A guide member and a sponge are located between the guide member and the magnetic part. A container with liquid is inserted into the tubular member and the needle penetrates the bottom of the container so that the liquid flows toward the protrusions which massage the user's head.

### BACKGROUND OF THE INVENTION

### 1. Fields of the Invention

The present invention relates to a massage device, and more particularly, to a head massage device to activate cutaneous nervous system.

### 2. Descriptions of Related Art

Life of the modern world today makes people have significant stress because of high competition and fast pace life. The busy life consumes too much energy, and people try to find a relief from the stress by massaging their head. The head nerves and cells need to be waked up and activated so as to activate the cutaneous nervous system and to adjust functions of the cerebral cortex to release pressure and stress. However, using fingers to massage cannot last long and only limited result can be expected. Therefore, a proper tool is needed.

The present invention intends to provide a head massage device to eliminate the shortcomings mentioned above.

### SUMMARY OF THE INVENTION

The present invention relates to a head massage device and comprises a head including a tubular member extending from the top thereof, and the tubular member includes a space which opens through the top end of the tubular member. A needle extends from the inner bottom of the tubular member. Multiple first bores are defined through the inner bottom of the tubular member. A room is defined in the underside of the head and a recess is defined in the inner top of the room. The first bores communicates with the recess. A handle is connected to the head and includes a vibrator received therein. A switch is located on the handle and electrically connected to the vibrator. A magnetic part is connected to the underside of the head and includes a recessed area. Multiple protrusions extend from the underside of the magnetic part and each protrusion includes a passage defined therethrough. Multiple holes are defined through the magnetic part, and each hole communicates with the passage of the protrusion located corresponding thereto. Multiple paths are defined in the inner end of the recessed area of the magnetic part and communicate between the holes.

A sponge is accommodated within the room of the head and the recessed area of the magnetic part. Multiple orifices are defined through the sponge. A guide member is located in the recess of the head and located on the sponge. Multiple second bores are defined through the top of the guide member. Multiple grooves are defined in the top surface and the bottom surface of the guide member. A container is removably inserted into the space of the tubular member. The container receives liquid therein. At least one seal ring is mounted to outside of the container.

Preferably, the recess is a conical-shaped recess, and the guide member is shaped to match the recess.

The advantages of the present invention is that the liquid can be massage oil, cream or medicine which is absorbed in the sponge, and the liquid is atomized by the vibration from the vibrator. The magnetic part generates magnetic energy so that when massaging the head by the protrusions, the cutaneous nervous system is activated.

The present invention will become more obvious from the following description when taken in connection with the accompanying drawings which show, for purposes of illustration only, a preferred embodiment in accordance with the present invention.

### BRIEF DESCRIPTION OF THE DRAWINGS

FIG. 1 is an exploded view of the head massage device of the present invention;

FIG. 2 is a perspective view to show the head massage device of the present invention;

FIG. 3 is a cross sectional view to show that the needle penetrates through the underside of the container, and

FIG. 4 is a cross sectional view to show that the container is slightly lifted, and the liquid in the container flows toward the protrusions.

### DETAILED DESCRIPTION OF THE INVENTION

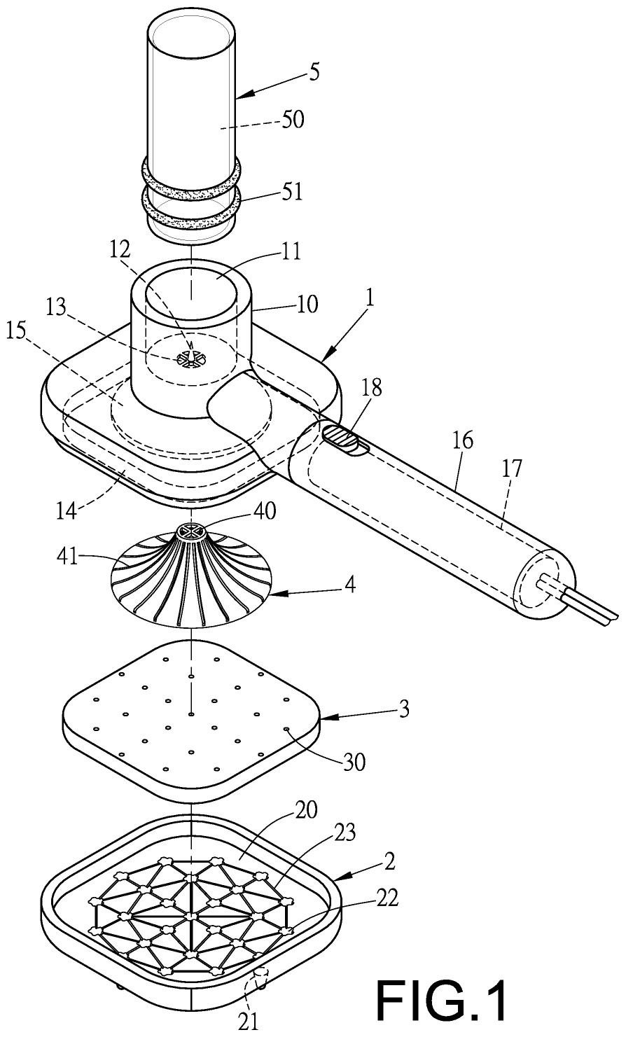

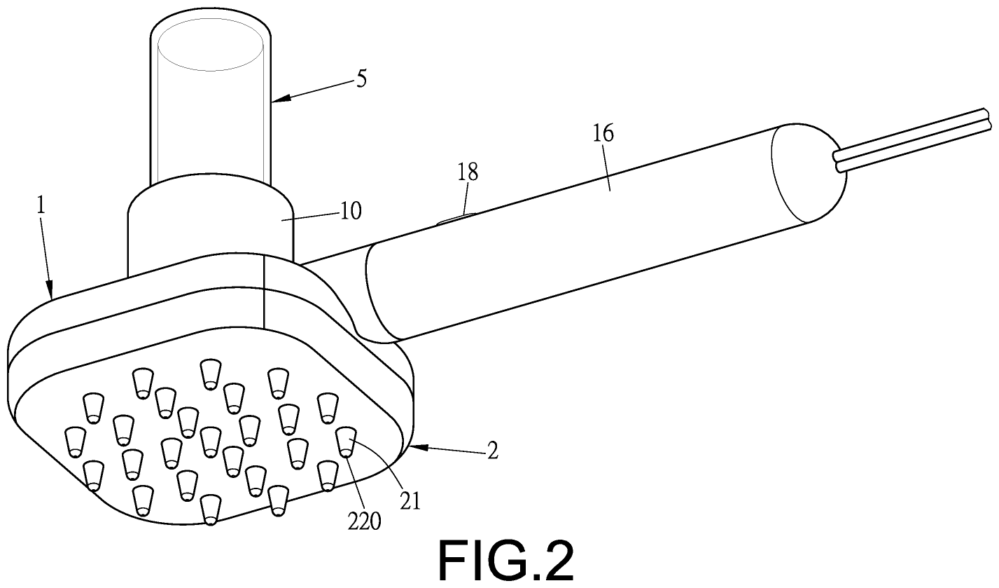

Referring to FIGS. 1 to 3, the head massage device of the present invention comprises a head 1 including a tubular member 10 extending from the top thereof. The tubular member 10 includes a space 11 which opens through the top end of the tubular member 10, and a needle 12 extends from the inner bottom of the tubular member 10. Multiple first bores 13 are defined through the inner bottom of the tubular member 10. A room 14 is defined in the underside of the head 1 and a recess 15 is defined in the inner top of the room 14. The first bores 13 communicate with the recess 15. The recess 15 is a conical-shaped recess.

A handle 16 is connected to the head 1 and includes a vibrator 17 received therein. A switch 18 is located on the handle 16 and electrically connected to the vibrator 17. A magnetic part 2 is connected to the underside of the head 1 and includes a recessed area 20 defined in the top thereof. Multiple protrusions 21 extend from the underside of the magnetic part 2 and are used to massage the user's head. Multiple holes 220 are defined through the magnetic part 2, and each hole 220 communicates with the passage 22 of the protrusion 21 located corresponding thereto. Each protrusion 21 has a passage defined therethrough. Multiple paths 23 are defined in the inner end of the recessed area 20 of the magnetic part 2 and communicate between the holes 220 so as to form a net-like system.

A sponge 3 is accommodated within the room 14 of the head 1 and the recessed area 20 of the magnetic part 2. Multiple orifices 30 are defined through the sponge 3. A guide member 4 is located in the recess 15 of the head 1 and located on the sponge 3. The guide member 4 is shaped to match the recess 15. Multiple second bores 40 are defined through the top of the protruded portion of the guide member 4. Multiple grooves 41 are defined in the top surface and the bottom surface of the guide member 4. A container 3 is removably inserted into the space 11 of the tubular member 10. The container 3 receives liquid 50 therein. Two seal rings 51 are mounted to outside of the container 3.

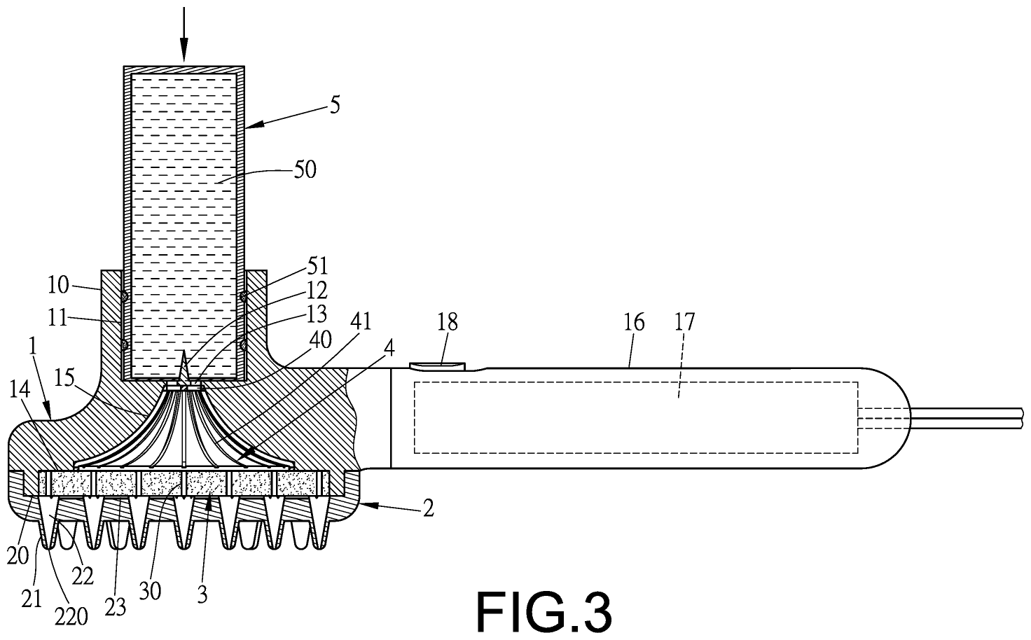

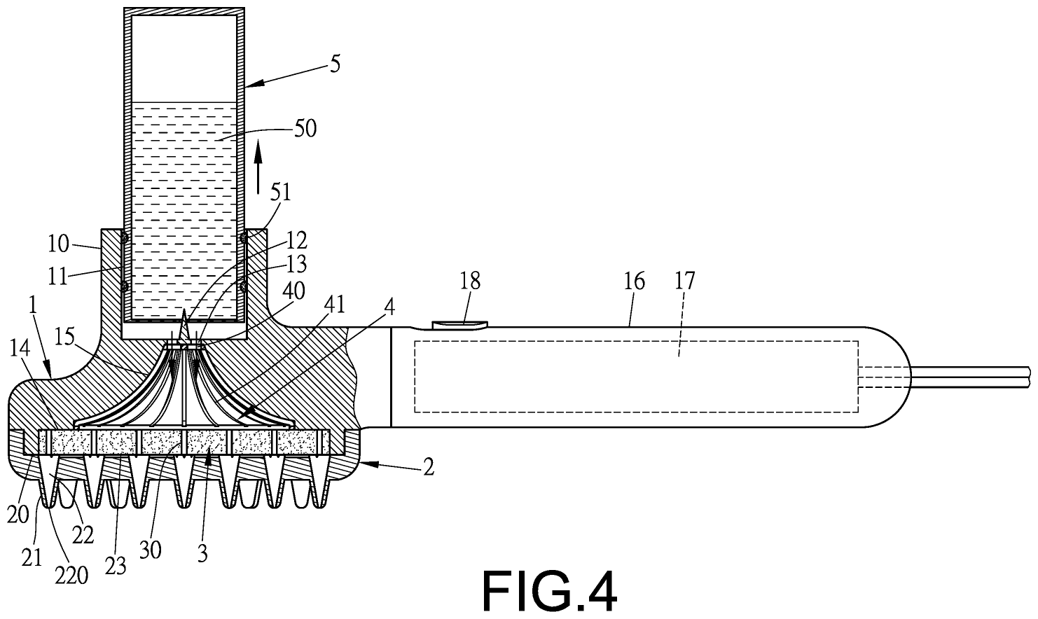

When in use, the container 5 is inserted into the space 11 of the tubular member 10 and the needle 12 penetrates through the bottom end the bottom of the container 5 as shown in FIG. 3. The container 5 is then slightly lifted as shown in FIG. 4, so that the liquid 50 in the container 5 flows through the first bores 13 of the tubular member 10, the second bores 40 of the guide member 4, the grooves 41 of the guide member 4 and is absorbed by the sponge 3. The user holds the handle 16 and puts the protrusions 21 of the magnetic part 2 on his/her head. The switch 18 is activated and the vibrator 17 vibrates. The liquid 50 in the sponge 3 is atomized by the vibration, and flows through the holes 220 of the magnetic part 2 and the passages 22 of the protrusions 21 to be applied to the user's head. The magnetic part 2 generates magnetic energy to adjust the magnetic field of our head. By the liquid 50 and the magnetic energy, and the use of the head massage device of the present invention, the cutaneous nervous system is activated, the functions of the cerebral cortex are adjusted, and the hair follicles are also stimulated. The liquid can be massage oil, cream or medicine according to practical needs.

While we have shown and described the embodiment in accordance with the present invention, it should be clear to those skilled in the art that further embodiments may be made without departing from the scope of the present invention.

What is claimed is:

1. A head massage device comprising:
a head including a tubular member extending from a top thereof, the tubular member including a space which opens through a top end of the tubular member, a needle extending from an inner bottom of the tubular member, multiple first bores defined through the inner bottom of the tubular member, a room defined in an underside of the head and a recess defined in an inner top of the room, the first bores communicating with the recess, a handle connected to the head and including a vibrator received therein, a switch located on the handle and electrically connected to the vibrator;
a magnetic part connected to the underside of the head and including a recessed area, multiple protrusions extending from an underside of the magnetic part and each protrusion including a passage defined therethrough, multiple holes defined through the magnetic part, each hole communicating with the passage of the protrusion located corresponding thereto, multiple paths defined in an inner end of the recessed area of the magnetic part and communicating between the holes;
a sponge accommodated within the room of the head and the recessed area of the magnetic part, multiple orifices defined through the sponge;
a guide member located in the recess of the head and located on the sponge, multiple second bores defined through a top of the guide member, multiple grooves defined in a top surface and a bottom surface of the guide member, and
a container removably inserted into the space of the tubular member, the container adapted to receive liquid therein, at least one seal ring mounted to outside of the container.

2. The head massage device as claimed in claim 1, wherein the recess is a conical-shaped recess, the guide member is shaped to match the recess.
